(19) United States
(12) Patent Application Publication
(10) Pub. No.: US 2018/0318340 A1
(43) Pub. Date: Nov. 8, 2018

(54) A ZEOLITE USED IN PLEURODESIS

(71) Applicant: YEDITEPE UNIVERSITESI, Istanbul (TR)

(72) Inventors: Ahmet AYDIN, Istanbul (TR); Mohammed CHAREHSAZ, Istanbul (TR); Hande SIPAHI, Istanbul (TR); Gulengul DUMAN, Istanbul (TR); Hayati CELIK, Istanbul (TR); Nurcan BAC, Istanbul (TR); Sina ERCAN, Istanbul (TR)

(73) Assignee: YEDITEPE UNIVERSITESI, Istanbul (TR)

(21) Appl. No.: 15/774,011

(22) PCT Filed: Mar. 15, 2016
(86) PCT No.: PCT/TR2016/050070

§ 371 Date: May 7, 2018

(30) Foreign Application Priority Data
Nov. 9, 2015 (TR) 2015/13993

(51) Int. Cl. A61K 33/06; A61K 9/00

(52) U.S. Cl. CPC A61K 33/06; A61P 41/00; A61K 9/0073

(57) ABSTRACT

The present invention relates to the use of a zeolite as a chemical agent with additional cytotoxic effects used in pleurodesis in the cases of liquid accumulation (pleural effusion) or air accumulation (pneumothorax) in the pleural cavity of lungs.

### CROSS REFERENCE TO THE RELATED APPLICATIONS

This application is the national phase entry of International Application No. PCT/TR2016/050070, filed on Mar. 15, 2016, which claims priority from Turkish Patent Application 2015/13993, filed on Nov. 9, 2015, the entire contents of which are incorporated herein by reference.

### TECHNICAL FIELD

The present invention relates to a zeolite used in pleurodesis which is performed in case of liquid accumulation (pleural effusion) or air accumulation (pneumothorax) in the pleural cavity of lungs.

### BACKGROUND

Pneumothorax is the accumulation of air between the visceral and parietal pleurae surrounding the lungs. When an individual with an existing pulmonary disease develops pneumothorax, it is called the secondary spontaneous pneumothorax. Pleurodesis is the medical procedure in which the pleural cavity is obliterated through adhesion of parietal and visceral pleurae which is achieved by introducing a sclerosing agent into the pleural cavity through a chest tube or thoracoscope. Pleurodesis aims to prevent the recurrence of pleural effusion and pneumothorax, and achieve full expansion of the lung. When tube thoracostomy fails, pleurodesis can be performed safely with low recurrence rates. It is also often required in lung cancer cases.

Various pharmaceuticals are used for pleurodesis. Among these pharmaceuticals, sterile talc powder is the most widely used one. It was first used by a surgeon named Norman Bethune in 1935 (Janssen, J P., Collier, G., Astoul, P., Tassi, G F., Noppen, M., Panadero-Rodriguez, F., Loddenkemper, R., Herth, F J F., Gasparini S., Marquette, C H., Becke, B., Froudarakis, M F., Driesen, P., Bolliger, C T., Tschopp, J M.: Safety of Pleurodesis with talc poudrage in malignant pleural effusion: a prospective cohort study. Lancet, 369:1535-39, 2007). The talc powder prepared sterilely and usually formulated to be 4 grams is administered to the pleura by a specialist in thoracic surgery. There are two types of administrations of talc powder. The first one is the introduction of 4 grams of sterile talc powder in suspension into the pleural cavity using a catheter while the second method involves spraying the talc powder in aerosol prepared in a pressure vessel into the pleura using a cannula.

### SUMMARY

The aim of the present invention is to enable the use of different types of zeolites in pleurodesis for the treatment of pulmonary diseases such as pleural effusions and pneumothorax.

Another aim of the present invention is to use powdered zeolite as an agent in pleurodesis.

Yet another aim of the present invention is to make use of the cytotoxic effect of zeolites on cancer cells.

### DETAILED DESCRIPTION

The present invention relates to the use of a zeolite as a chemical agent in pleurodesis procedures performed in pneumothorax and/or pleural effusions. In order to realize the aim of the present invention, synthetic zeolite (zeolite X and zeolite Y) and/or natural zeolite (clinoptilite) may be used.

Due to its cytotoxic effect, zeolite also kills cancer cells in the site of administration in lung cancer cases. Thus, it has a dual effect as both a pleurodesis chemical agent and cytotoxic agent in the treatment of pleural effusions and/or pneumothorax secondary to lung cancer, as well as in the treatment of lung cancer itself. The chemical composition of the relevant zeolite, which is of mineral origin, is hydrated aluminum silicate.

The preferred embodiment of the invention is to introduce the powdered zeolite suspension into the pleural cavity through a catheter. Most preferably, the diameter of a powdered zeolite particle is approximately 20 micrometers.

In another embodiment of the invention, the powdered zeolite material is sprayed into the pleural cavity through aerosol tubes during pleurodesis.

1. A method of performing pleurodesis procedures performed in cases of pneumothorax and/or pleural effusion, comprising utilizing a type of zeolite as a chemical agent.

2. A method of performing pleurodesis procedures in pleural effusions and/or pneumothorax secondary to lung cancer, comprising utilizing a type of zeolite as a pleurodesis chemical agent and a cytotoxic agent, wherein the zeolite is in a form of hydrated aluminum silicate.

3. The method of claim 1, further comprising introducing the type of zeolite into a pleural cavity using a catheter, wherein the type of zeolite is in suspension form.

4. The method of claim 1, further comprising introducing the type of zeolite into a pleural cavity using aerosol tubes, wherein the type of zeolite is in powdered form.

5. The method of claim 2, further comprising introducing the type of zeolite into a pleural cavity using a catheter, wherein the type of zeolite is in suspension form.

6. The method of claim 2, further comprising introducing the type of zeolite into a pleural cavity using aerosol tubes, wherein the type of zeolite is in powdered form.
